(19) United States
(12) Patent Application Publication
(10) Pub. No.: US 2012/0194661 A1
(43) Pub. Date: Aug. 2, 2012

(54) ENDSCOPIC SPECTRAL DOMAIN OPTICAL COHERENCE TOMOGRAPHY SYSTEM BASED ON OPTICAL COHERENT FIBER BUNDLE

(75) Inventors: Byeongha Lee, Buk-gu (KR); Jonghyun Eom, Buk-gu (KR); Woo June Choi, Buk-gu (KR); Eun Jung Min, Buk-gu (KR)

(73) Assignee: GWANGJU INSTITUTE OF SCIENCE AND TECHNOLOGY, Buk-gu (KR)

(21) Appl. No.: 13/295,794

(22) Filed: Nov. 14, 2011

(30) Foreign Application Priority Data
Dec. 24, 2010 (KR) 10-2010-0134640

(51) Int. Cl. A61B 1/04; H04N 7/18; A61B 1/07

(52) U.S. Cl. 348/68; 348/E07.085

(57) ABSTRACT

The present invention relates to a spectral domain optical coherence tomography apparatus having an endoscopic small-sized probe, and more particularly, to a technology imaging an external shape or an internal structure of a sample by a non-contact and non-invasive method by applying an optical coherent fiber bundle probe attached with a lens to Michelson interferometer or a Fizeau interferometer.

### CROSS-REFERENCE TO RELATED APPLICATION

This application claims under 35 U.S.C. §119(a) the benefit of Korean Patent Application No. 10-2010-0134640 filed on Dec. 24, 2010, the entire contents of which are incorporated herein by reference.

### BACKGROUND

(a) Technical Field

The present invention relates to a spectral domain optical coherence tomography apparatus having an endoscopic small-sized probe, and more particularly, to a technology imaging an external shape or an internal structure of a sample by a non-contact and non-invasive method by applying an optical coherent fiber bundle probe attached with a lens to Michelson interferometer or a Fizeau interferometer.

(b) Background Art

In recent years, a low coherence interferometry (LCI) adopting a principle of a Michelson interferometer has been developed in order to acquire a surface shape and an internal structure of a sample by using light. In a routine system, 1-dimension and 2-dimension lateral scanning of a sample stage is required to implement 2D and 3D images and in the case of a temporal domain interferometer, longitudinal scanning a reference stage is also additionally required. A scanner for the sample stage is generally configured in a bulk form by using a Galvano mirror and a lens and a research into the miniaturization of the probe for internal imaging of a human body in an endoscope and a catheter has also been actively progressed.

In order to manufacture a small-sized probe suitable for an endoscope type, a complicated scanner constituted by an MEMS based small-sized mirror and line, a rotary motor, a piezoelectric element, a lens system, and the like placed at the end of the probe was used in the related art. However, when the MEMS mirror and line or rotary motor is used at the end of the probe, a manufacturing process is very complicated and a manufacturing cost is also significantly consumed. In addition, an additional power supplying apparatus is required for the operation, such that the volume of the scanner increases and the flexibility of the probe deteriorates and an expected accident may occur as power is supplied in the human body. Further, bulk elements such as a microprism or a reflector lens are generally used to irradiate light to the sample or collect reflected light. In the bulk elements, accurate optical-axis alignment between optical fibers and a lens system is required and optical loss increases as the number of optical elements constituting a lens system increases.

Meanwhile, in an endoscopic LCI system in the related art, two different optical paths (the sample stage and the reference stage) are formed in an interferometer to generate an interference signal. However, in the case of using different optical paths in one interferometer, the interference signal is very vulnerable to a temperature change, external disturbances such as the flow of air, vibration, and the like and the polarization difference between the reference stage and the sample stage should be adjusted at the time of acquiring the interference signal. Accordingly, two optical paths constituting the interferometer need to be the same as each other as possible.

### SUMMARY OF THE DISCLOSURE

The present invention has been made in an effort to provide an optical tomography system with an endoscopic probe, which minimizes the size of the probe by maximally simplifying the structure of the end of the probe and which is easy to handle, excellent in flexibility, and easy to manufacture. To this end, in the present invention, by a common path interferometer type using a sample stage and a reference stage as one path by using an optical coherent fiber bundle, the size of the entire system is minimized and the distortion of an image signal generated by the sample stage and the reference stage that are separated from each other is minimized.

According to an exemplary embodiment of the present invention, there is provided an optical coherence tomography image acquiring method for acquiring a tomography image of a sample surface and an internal structure based on an optical fiber bundle, including: splitting and irradiating a light source having a predetermined bandwidth based on a center wavelength into a fixed reference stage and a sample stage constituted by an optical fiber bundle through an optical splitter; generating an interference signal after light reflected on a mirror of the reference stage and light reflected on the sample through the optical splitter again through the optical filter bundle meet each other again; perform 1D lateral scan with respect to an incident surface of the sample stage constituted by the optical fiber bundle in order to acquire 2D image information on the sample and detecting interference signals generated from light reflected on the sample surface and an internal tomography interface layer by using a spectrometer of a detection stage and a line CCD camera; and acquiring a tomography image on after signal processing the detected interference signals and outputting the acquired tomography image onto a monitor as a video.

According to exemplary embodiments of the present invention, in an optical coherent fiber bundle probe which is suitable to be used as an endoscopic probe in an optical tomography system is provided, flexibility as the probe can be maximized by minimizing a configuration of the end of a probe to be inserted into a human body by scanning an optical coherent fiber bundle incident surface. Further, by substituting a Michelson interferometer type used in an existing optical tomography system with a Fizeau interferometer type, a sample stage and a reference stage are used as one path to reduce the distortion of an image due to the difference between the both stages, thereby acquiring a clear image. As a result, it is expected that the exemplary embodiments of the present invention will be adopted in an endoscopic micro medical image diagnosis which has been actively progressed in recent years.

### BRIEF DESCRIPTION OF THE DRAWINGS

FIGS. 1A-B are a schematic diagram of a system configured by using a spectral domain optical coherence interferometer based on an optical coherent fiber bundle according to an exemplary embodiment of the present invention;

FIG. 1A is a schematic diagram of a spectral domain optical coherence system based on a Michelson interferometer based optical coherent fiber bundle according to an exemplary embodiment of the present invention;

FIG. 1B is a schematic diagram of a spectral domain optical coherence system based on an optical coherent fiber bundle, which is configured to use a reference stage and a sample stage as one path according to an exemplary embodiment of the present invention;

FIG. 2 is a schematic diagram of a sample stage of a spectral domain optical coherence interferometer system based on an optical coherent fiber bundle according to an exemplary embodiment of the present invention;

FIG. 3 is a mimetic cross-sectional view of an optical coherent fiber bundle used in the spectral domain optical coherence interferometer system based on an optical coherent fiber bundle according to the exemplary embodiment of the present invention;

FIG. 4 is a real cross-sectional view of the optical coherent fiber bundle used in the spectral domain optical coherence interferometer system based on an optical coherent fiber bundle according to the exemplary embodiment of the present invention;

FIGS. 5A-B are a photograph picked up on an optical coherent fiber bundle emission surface after making light be incident in only one optical fiber among several optical fibers constituting an optical coherent fiber bundle by focusing light through an object lens on the optical coherent fiber bundle used in the spectral domain optical coherence interferometer system based on an optical coherent fiber bundle according to the exemplary embodiment of the present invention and a graph showing the intensity emitted from the emission surface;

FIG. 5A is the photography actually picked up on the optical coherent fiber bundle emission surface;

FIG. 5B is the graph showing the intensity of light emitted from the optical coherent fiber bundle emission surface;

FIGS. 6A-B show an interference spectrum actually acquired by using the spectral domain optical coherence interferometer system based on an optical coherent fiber bundle according to the exemplary embodiment of the present invention and a depth information signal regarding a sample acquired by Fourier-transforming the acquired interference spectrum signal;

FIG. 6A shows the interference spectrum signal acquired by measuring a real sample;

FIG. 6B shows the depth information signal regarding the sample acquired by Fourier-transforming the interference spectrum signal of FIG. 6A;

FIGS. 7A-B show a tomography image of a sample constituted by a slide glass and a metal-deposited mirror acquired through an apparatus of the present invention and a depth information graph of a sample acquired by Fourier-transforming an interference spectrum signal;

FIG. 7A shows the tomography image of the sample constituted by the slide glass and the metal-deposited mirror acquired through the apparatus of the present invention;

FIG. 7B shows a 1-dimension depth information graph acquired at a predetermined location of the sample;

FIGS. 8A-B show a tomography image of a sample constituted by two stacked slide glasses and a metal-deposited mirror acquired through an apparatus of the present invention and a depth information graph of a sample acquired by Fourier-transforming an interference spectrum signal;

FIG. 8A shows the tomography image of the sample in which two slide glasses are stacked on one metal-deposited mirror acquired through the apparatus of the present invention;

FIG. 8B shows the 1-dimension depth information graph acquired at the predetermined location of the sample;

FIGS. 9A-B are a mimetic diagram of a sample stage in which a uniaxial Galvano scanning mirror and a uniaxial linear feeding apparatus to the spectral domain optical coherence system based on an optical coherent fiber bundle according to the exemplary embodiment of the present invention to enable 2D tomography imaging;

FIG. 9A is the mimetic diagram of the sample stage in which the uniaxial Galvano scanning mirror is applied to the spectral domain optical coherence system based on an optical coherent fiber bundle;

FIG. 9B is the mimetic diagram of the sample stage in which the uniaxial linear feeding apparatus is applied to the spectral domain optical coherence system based on an optical coherent fiber bundle;

FIGS. 10A-B are a mimetic diagram of a sample stage in which a biaxial Galvano scanning mirror and a biaxial linear feeding apparatus is applied to the spectral domain optical coherence system based on an optical coherent fiber bundle according to the exemplary embodiment of the present invention to enable 3D tomography imaging;

FIG. 10A is the mimetic diagram of the sample stage in which the biaxial Galvano scanning mirror is applied to the spectral domain optical coherence system based on an optical coherent fiber bundle;

FIG. 10A is the mimetic diagram of the sample stage in which the biaxial linear feeding apparatus is applied to the spectral domain optical coherence system based on an optical coherent fiber bundle;

FIG. 11 shows an exemplary embodiment in which a green lens is attached to the end of an optical coherent fiber bundle of a sample stage in a spectral domain optical coherence interferometer based on an optical coherent fiber bundle according to the present invention;

FIG. 12 shows an exemplary embodiment in which an optical fiber integrated lens is formed at a front end of an optical coherent fiber bundle in order to focus or collect a large light amount on the end of the optical coherent fiber bundle of the sample stage in the spectral domain optical coherence interferometer based on an optical coherent fiber bundle according to the present invention;

FIG. 13 shows an exemplary embodiment in which a coreless silica fiber (CSF) is coupled to the front end of the optical coherent fiber bundle by using an optical fusion connection method and thereafter, the optical fiber integrated lens is formed at a front end of the CSF in order to focus or collect a large light amount on the end of the optical coherent fiber bundle of the sample stage in the spectral domain optical coherence interferometer based on an optical coherent fiber bundle according to the present invention;

FIG. 14 shows an exemplary embodiment in which an optical fiber integrated lens vertically cut to enable side imaging is formed at the front end of the optical coherent fiber bundle in order to focus or collect a large light amount on the end of the optical coherent fiber bundle of the sample stage in the spectral domain optical coherence interferometer based on an optical coherent fiber bundle according to the present invention;

FIG. 15 shows an exemplary embodiment in which a 3D image is implemented by rotating the probe of FIG. 14 in which the optical fiber integrated lens vertically cut to enable side imaging is formed at the front end of the optical coherent fiber bundle in order to focus or collect a large light amount on the end of the optical coherent fiber bundle of the sample stage in the spectral domain optical coherence interferometer based on an optical coherent fiber bundle according to the present invention; and

FIG. 16 shows another exemplary embodiment in which a micro focusing lens is installed and packaged at the end of an optical coherent fiber bundle of a sample stage in a spectral domain optical coherence interferometer based on an optical coherent fiber bundle according to the present invention.

### DETAILED DESCRIPTION

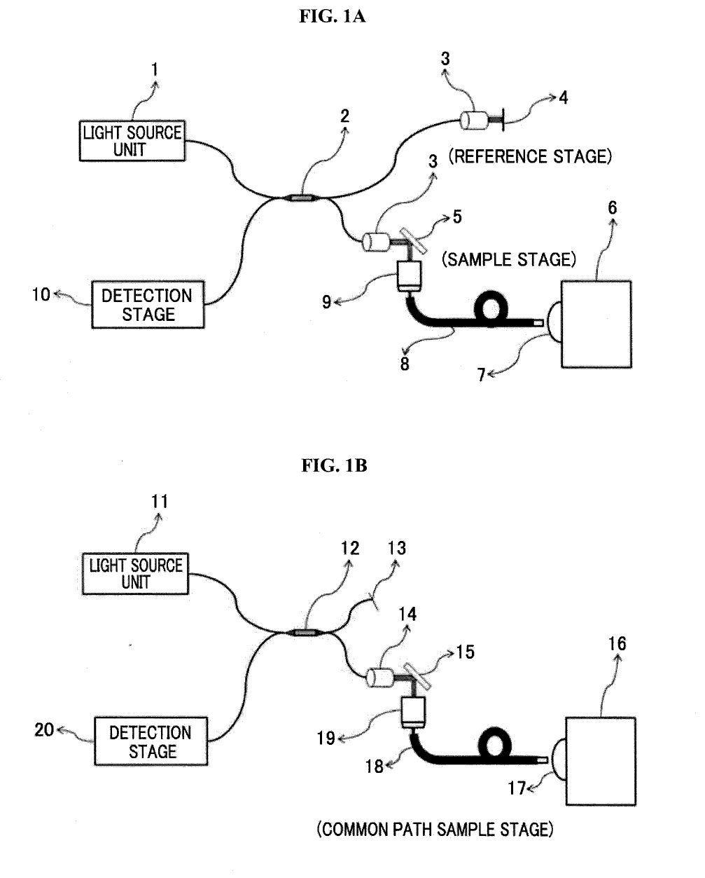

FIG. 1 is a schematic diagram of a spectral domain optical tomography system based on an optical coherent fiber bundle which can be manufactured in an endoscopy type according to an exemplary embodiment of the present invention. The system may be configured in two types. FIG. 1A is a schematic diagram of a first system. Each of a detection stage constituted by a light source unit 1 of a light source having a predetermined center wavelength and a predetermined bandwidth, a collimator, a focusing lens, and a line CCD camera, a sample stage capable of placing a sample to be measured and changing the position of the sample, and a reference stage constituted by a mirror 4 and a beam balancer 3 is connected to a 50:50 beam splitter 2. FIG. 1B shows a second system as a spectral domain optical tomography system based on an optical coherent fiber bundle, which has a schematic diagram similarly as the first system but has a common path structure in which the reference stage and the sample stage are coupled to each other. The second system uses the same light source unit 11 as the first system and the detection stage 20 is also constituted by the beam balancer, the focusing lens, and the line CCD camera similarly as the first system. The light source unit, the sample stage, and the detection stage are connected with each other by the beam splitter of which one port is blocked.

The first system, which is an optical coherence imaging system using an optical coherent fiber bundle as an endoscopic probe, basically includes a light source unit 1, a detection stage 10, a sample stage, and a reference stage. The basic structure of the system uses a Michelson interferometer and a light source has a center wavelength of 830 nm and a bandwidth 60 nm. Light emitted from the light source is split into the reference stage and the sample stage at a ratio of 50:50 by the beam splitter 2. Light split into the sample stage is irradiated to the sample through an optical fiber and light reflected or scattered on a sample surface and an internal layer is inputted through the optical fiber again. Light split into the reference stage of the system is also reflected on the mirror 4 of the reference stage to be inputted into the optical fiber again and merged by the beam splitter 2 to form an interference signal. The interference signal has a spatial frequency determined by the optical path difference between the light emitted from the sample stage and the reference stage on a wavelength spectrum, and as a result, the interference signal is dispersed into a component for each wavelength through a spectrometer of the detection stage 10 to be detected by the line CCD camera. The detected signal is restored to the surface and an internal image through frequency analysis and is displayed on a computer monitor.

FIG. 1B is a schematic diagram of the second system that is a common path optical coherence imaging system using the optical coherent fiber bundle. The second system includes a light source unit 11, a detection stage 20, and a common path sample stage. The second system is configured by a Fizeau interferometer having a common path sample stage in which the sample stage and the reference stage are coupled as one. The reference stage and the sample stage for generating the interference signal are included in one optical coherent fiber bundle. The light emitted from the light source is split by the 50:50 beam splitter in the first system, but reference stage light and sample stage light in the second system are formed by light reflected on the emission surface of the optical coherent fiber bundle and light reflected on the sample, respectively. Light is transmitted by using a wideband light source 11 and a single-mode optical fiber and detected by a beam splitter 12. Light which is incident in the beam splitter 12 of which one side is blocked is irradiated to only an optical coherent fiber bundle 18. The light irradiated to the optical coherent fiber bundle 18 is converted into a parallel light by a beam balancer 14 and is incident in a Galvano scanning mirror 15. An objective lens 19 is used in order to focus light reflected on the Galvano scanning mirror 15 on a predetermined core of the optical coherent fiber bundle. The Galvano scanning mirror 15 scans the light on an incident surface of the optical coherent fiber bundle in order to generate a 2D image. Light incident through the objective lens 19 is scanned and focused on each one optical coherent fiber bundle core. The focused light is transmitted to the emission surface of the optical coherent fiber bundle through the optical coherent fiber bundle and is incident in a sample 17 positioned on a sample stage 16 by passing through the emission surface of the optical coherent fiber bundle again. The beam splitter 12 in the system may be substituted with even an optical circulator without the need block one side of the beam splitter.

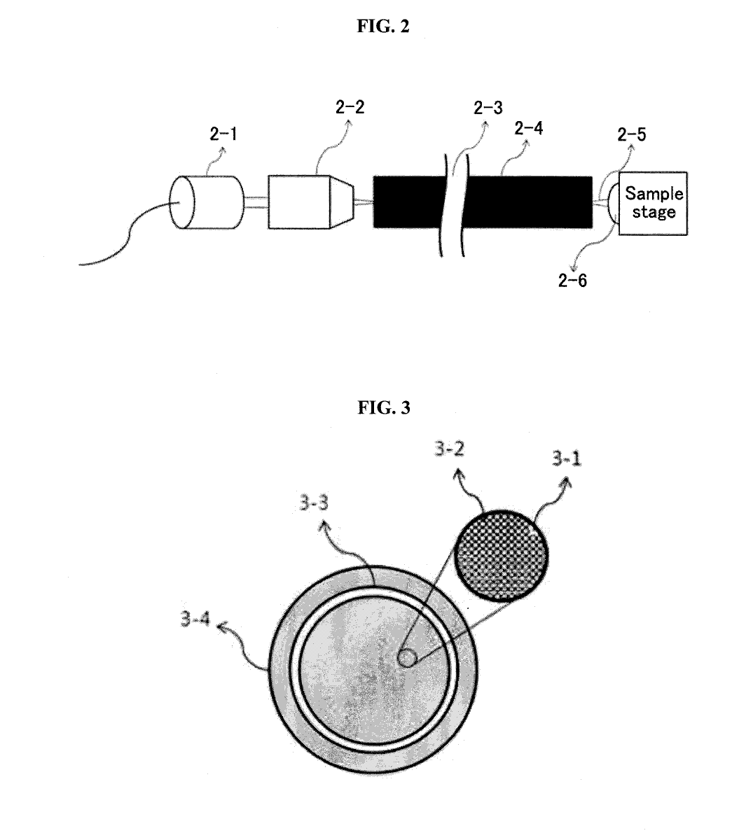

FIG. 2 shows a probe of the sample stage in the optical coherence imaging system using the optical coherent fiber bundle as the endoscopic probe. Light is transmitted to a beam balancer 2-1 through a single core optical fiber and the light passing through the beam balancer becomes parallel light to be projected by an objective lens 2-2 and the light passing through the objective lens 2-2 is focused on one core of an optical coherent fiber bundle 2-4 which is positioned at a focus distance of the objective lens 2-2. The focused light is transmitted by one core constituting the optical coherent fiber bundle 2-4 to be sent to a sample 2-6 on a sample stage 2-7. Light 2-5 emitted from the emission surface of the optical coherent fiber bundle is projected to the sample 2-6 and the light reflected or scattered on the sample is focused through the optical coherent fiber bundle again, such that light is irradiated in an opposite direction to the incident direction. The light reflected or scattered on the sample surface and the internal layer is irradiated through the optical fiber again and is coupled with the light reflected on the emission surface of the optical coherent fiber bundle, which serves as the reference stage light in the first system to form interference. The interference signal is detected by the detection stage that plays the same role in the first system. The interference signal has a difference spatial frequency component by the optical path difference of the light reflected or scattered and the interference signal is dispersed into a component for each wavelength through the spectrometer of the detection stage to be detected by the line CCD camera. The detected signal is restored to the surface and the internal image through frequency analysis and displayed on the computer monitor. The sample stage and the reference stage that are separated from each other which are required in the first system may be coupled as one stage in the second system to reduce an additional cost when the system is manufactured and has an advantageous in stabilization and miniaturization of the system to configure a low-priced miniaturized system. Further, a bulk optical lens system and an additional system required in the related art are simplified through the system and an image in which optical loss is reduced and a signal to noise ratio (SNR) is improved can be acquired.

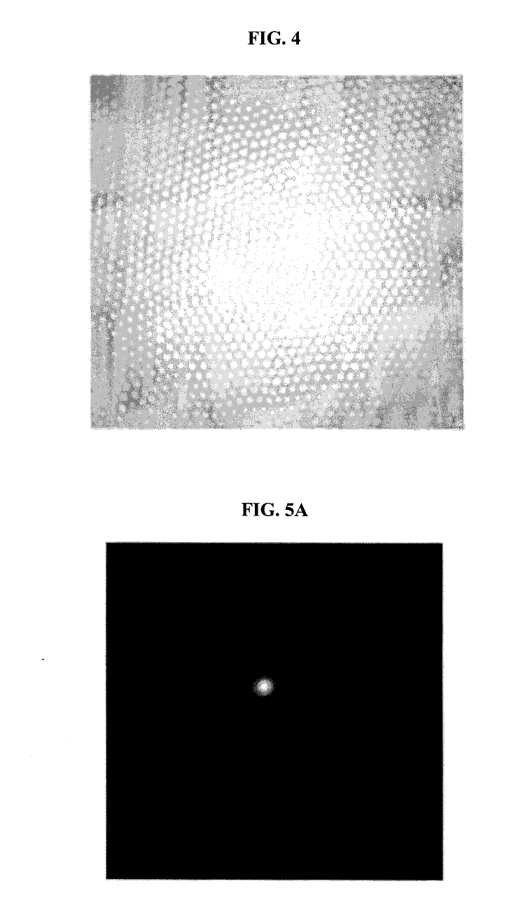

The optical coherent fiber bundle used in the present invention is a kind of a special optical fiber that transfers an image projected onto one surface of the bundle to an opposite surface without the distortion of the image. In the present invention, an optical coherent fiber bundle in which ten thousands of cores are arranged in one cladding at regular intervals is used. On a cross section of the optical coherent fiber bundle, ten thousands of cores 3-2 are arranged in one cladding 3-1 at a predetermined arrangement, in the optical coherent fiber bundle, as shown in FIG. 3. In this case, the optical coherent fiber bundle has a diameter in the range of 0.4 to 2 mm, 10000 to 100000 cores are focused on one cladding, and the cores may be arranged at regular intervals with the distance between the cores, which is within 4 μm. In order to protect the core 3-2 and the cladding 3-1, the core 3-2 and the cladding 3-1 are surrounded by a silica jacket 3-3. In addition, a plastic coating 304 which is thicker than the silica jacket 3-3 is configured for secondary protection. FIG. 4 is a diagram by picking up an actual cross-sectional photograph used in the present invention acquired by using a fiber optic video inspector. It can be seen that the cores are arranged in cladding at regular intervals.

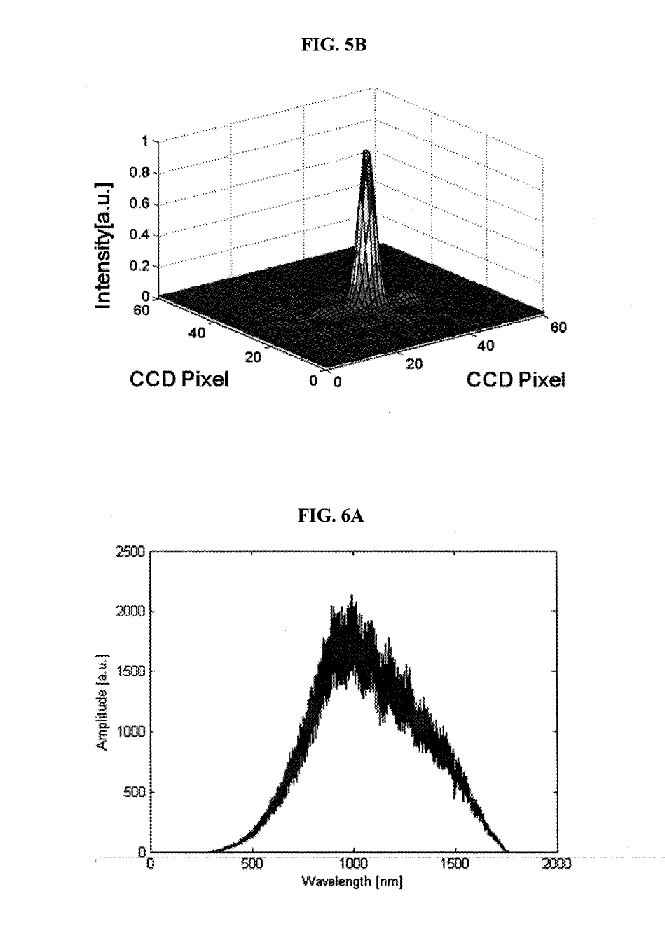

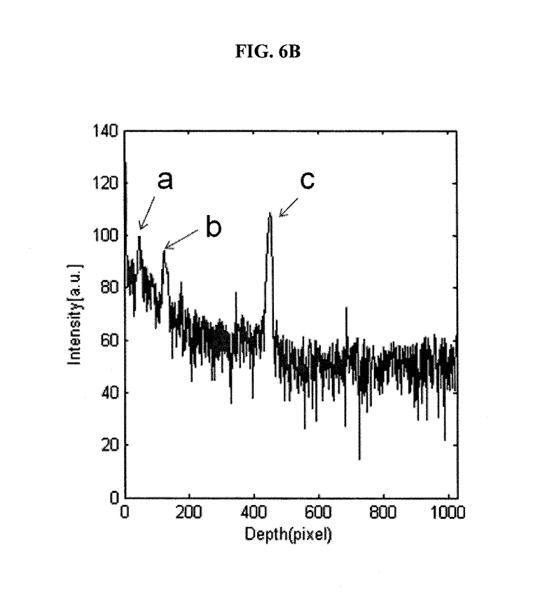

FIG. 5 is a result of measuring the light emitted from the optical coherent fiber bundle emission surface when light focused on the optical coherent fiber bundle through the objective lens is transmitted through the optical coherent fiber bundle. FIG. 5A is a photograph showing the case where light focused on one predetermined core among several cores constituting the optical coherent fiber bundle is emitted through the optical coherent fiber bundle emission surface, which is acquired by using the CCD camera. In FIG. 5A, a white circle represents light emitted from one core of the optical coherent fiber bundle. FIG. 5B is the graph showing the intensity distribution of light emitted from the optical coherent fiber bundle emission surface. The light emitted from one core of the optical coherent fiber bundle serves as one pixel configuring an image when the image is formed. Tomography information may be acquired through interference spectrum signal analysis acquired by the detection stages of the two systems. FIG. 6A shows an interference spectrum acquired through the detection stage. The finally acquired interference spectrum signal may be expressed by Equation 1.

$$I = {{\sum }^{j}}_{i} [ {I}_{D C} ( i ) + A ( i ) \times {\sum }_{m} cos ( k ( i ) \times n \times {Vz}_{m} ] \qquad [ Equation 1 ]$$

(Bandwidth of section [i,j] light source, m=0, 1, 2, L)

Herein, I_{DC}(i) is removed as unnecessary information when an actual tomography image is implemented with a signal which is irrelevant to interference, that is, has no interference in an interference signal acquired in the detection stage. A(i) is determined by the shape of a light source used to determine an envelope of the interference spectrum signal. k(i) as a wave number has a relationship of k=2π/λᵢ, and λᵢ represents each wavelength in a light source bandwidth. In addition, n represents and Vzₘ represents the optical path difference between the reference stage and tomography interfaces in the sample, i.e., the depth information of the sample. The interference spectrum signal is transmitted to a signal having only depth information through Fourier transformation to acquire the internal tomography information of the sample. FIG. 6B represents the tomography depth information of the sample acquired by Fourier-transforming the interference spectrum signal acquired by the detection stage. In FIG. 6B, signals a and b represent depth information on interference signals generated on front and rear surfaces of a micro-slide glass as a result acquired by measuring the tomography image while putting the micro-slide glass on the sample stage. Signal c as an unnecessary signal generated by the interference between the end of the objective lens and the end of the optical coherent fiber bundle in which light of the objective lens is incident in the sample stage of FIG. 2 may be removed by adjusting optical alignment.

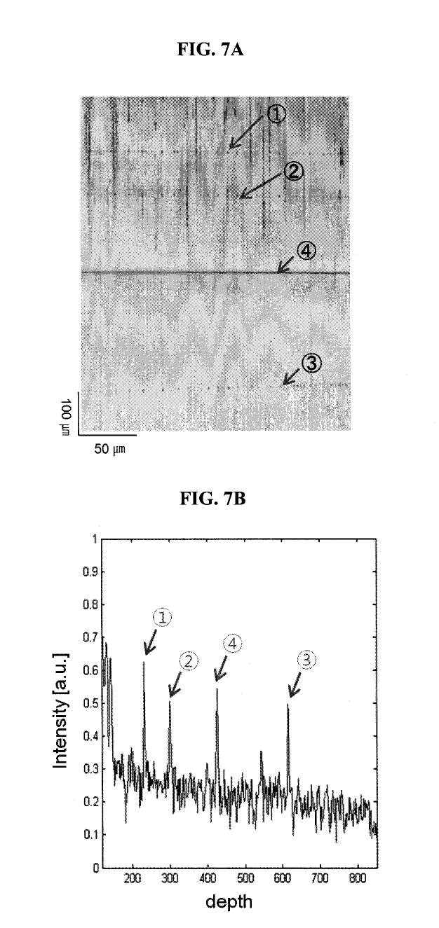

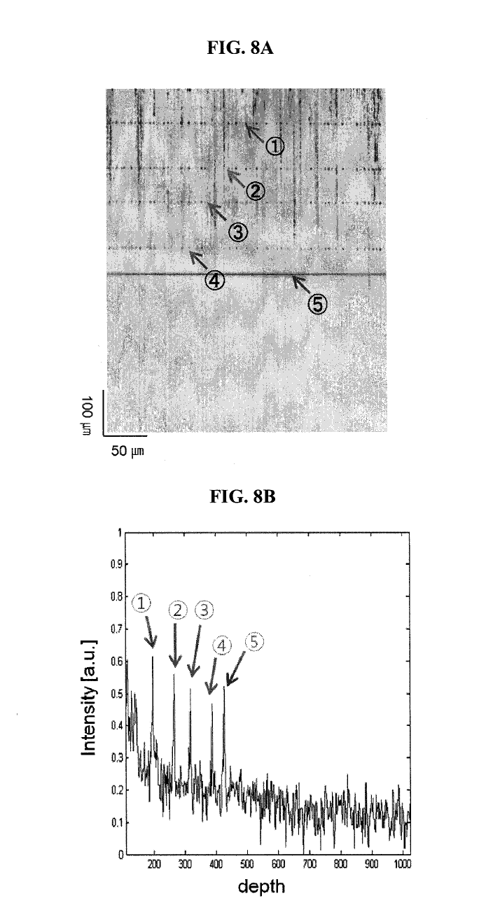

In each of FIGS. 7 and 8, 2D depth information on a predetermined position is acquired by using a tomography image of the sample acquired by the second system of the present invention. The 2D depth information is acquired by accumulating 1D depth information. As the sample used in FIG. 7, the micro-slide glass is put on the metal-deposited mirror and as the sample used in FIG. 8, two micro-slide glasses are stacked. FIG. 7A shows the acquired sample tomography image of which the size is 0.75 mm×0.35 mm. As shown in FIG. 7A, reflection surfaces (an upper surface 1 of the micro-slide glass and a lower surface 2 of the micro-slide glass) of the micro-slide glass which is the interface of the sample and a gold mirror surface can be discriminated from each other. FIG. 7B is a graph showing the depth information of the sample acquired by Fourier transforming the interference spectrum signal of the sample. Signals 1, 2, and 3 are generated by the upper surface, the lower surface, and the gold mirror surface of the micro-slide glass, respectively. FIG. 8 further shows the tomography image of the sample of which the size is 0.75 mm×0.35 mm. As shown in FIG. 8A, reflection surfaces (an upper surface 1 of the micro-slide glass and a lower surface 2 of the micro-slide glass) of a first micro-slide glass and reflection surfaces (an upper surface 3 of the micro-slide glass and a lower surface 4 of the micro-slide glass) of a second micro-slide glass which are the interfaces of the sample can be discriminated from each other. FIG. 8B is a graph showing the depth information of the sample acquired by Fourier transforming the interference spectrum signal of the sample. Signals 1, 2, 3, and 4 are each generated by the upper surface and the lower surface of the first micro-slide glass and the upper surface and the lower surface of the second micro-slide glass, respectively. It can be seen that an air layer is provided between the first micro-slide glass and the second micro-slide glass. Signal 4 of FIG. 7 and signal 5 of FIG. 8 may be generated by adjusting optical alignment through the interference between the end of the objective lens and the end of the optical coherent fiber bundle in which the light of the objective lens in the sample stage of FIG. 2. Therefore, it is seen that tomography information of the sample can be acquired by using the system of the present invention through FIGS. 7 and 8.

In the present invention, various probes may be configured in the optical coherent fiber bundle of the sample stage by using various optical equipments and feeding apparatuses so as to implement the miniaturization of the probe required in the existing endoscopic optical coherence imaging system using the optical coherent fiber bundle.

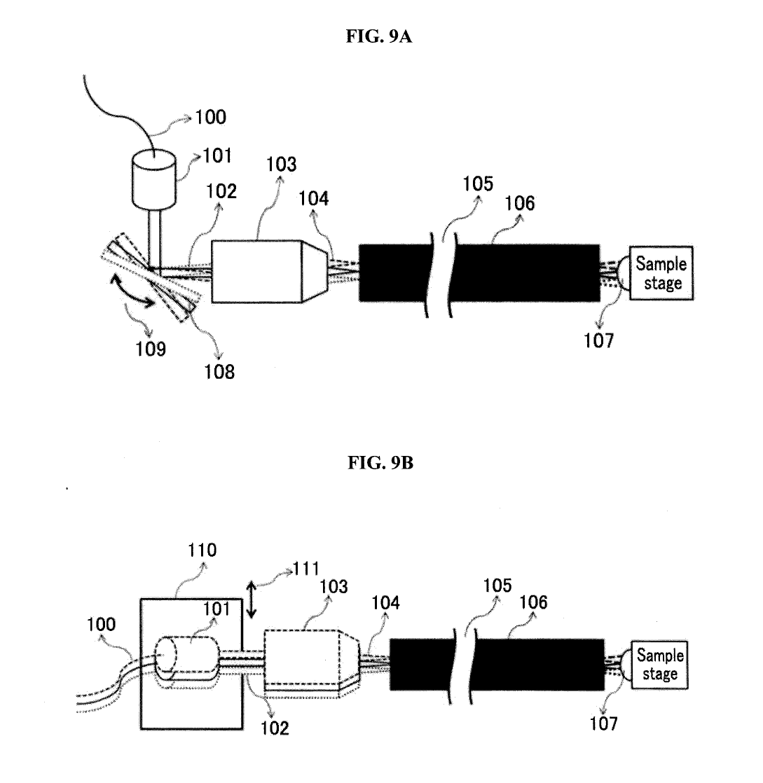

In a first exemplary embodiment, a basic optical coherence imaging system includes a beam balancer 101, an objective lens 103, an optical coherent fiber bundle 106, a scanning mirror 109, and a sample stage 108. As shown in FIG. 9A, a Galvano scanning mirror 10 needs to rotate in order to acquire a tomography image of a predetermined region of the sample. The scanning mirror 108 rotates in the same direction as 119 to change a path of light emitted from the beam balancer 101, which is incident in the objective lens 103. The changed path of light is scanned through line movement in the optical coherent fiber bundle to be focused sequentially on the plurality of cores positioned in the optical coherent fiber bundle to form the 2D image for the sample.

In a second exemplary embodiment, FIG. 9A shows a schematic diagram, but as shown in FIG. 9B, the length of a predetermined section is scanned in the optical coherent fiber bundle in the same direction as 111 by using not rotating movement of the Galvano scanning mirror 108 but a linear-direction feeding apparatus 110 in the method of changing the path of light in order to form the 2D image as shown in FIG. 9B. Light focused on the cores in the optical coherent fiber bundle is transmitted to be projected to the sample, thereby forming the 2D image.

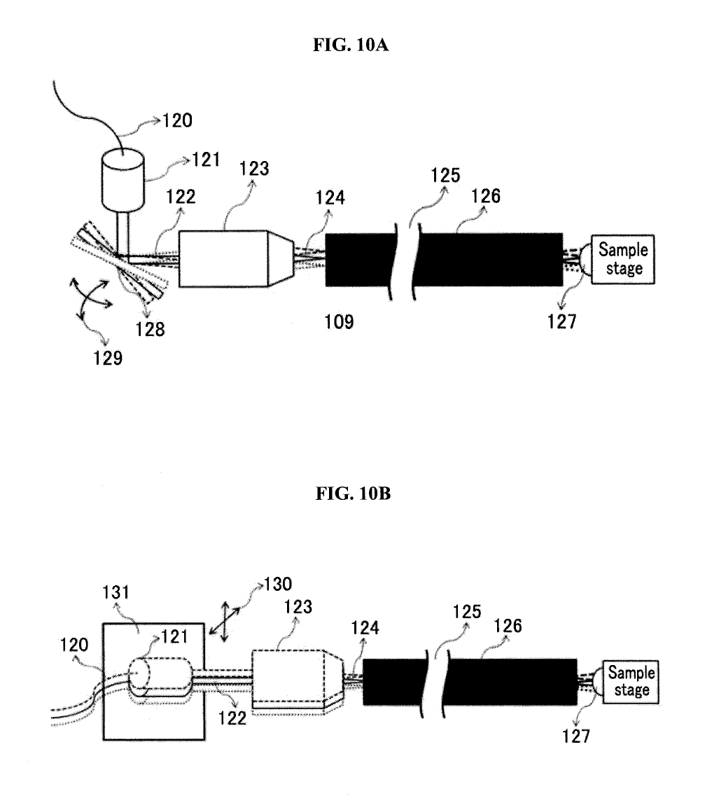

As shown in FIG. 10A, in a third exemplary embodiment, a biaxial Galvano scanning mirror 128 is additionally used in order to acquire a 3D tomography image of a sample in an existing sample stage (FIG. 9A) for forming a 2D tomography image. A range of a predetermined section is scanned in the optical coherent fiber bundle in the same direction as 129 by using the biaxial Galvano scanning mirror 128. Light focused on the optical coherent fiber bundle core within the predetermined section is transmitted and projected to the sample within the predetermined section to form the 3D image.

As shown in FIG. 10B, in a fourth exemplary embodiment, a bidirectional linear Galvano scanning mirror 131 is additionally used in order to acquire a 3D tomography image of a sample in an existing sample stage (FIG. 9B) for forming a 2D tomography image. A range of a predetermined section is scanned in the optical coherent fiber bundle in the same direction as 130 by using the bidirectional linear feeding apparatus 131. Light focused on the optical coherent fiber bundle core within the predetermined section is transmitted and projected to the sample within the predetermined section to form the 3D image.

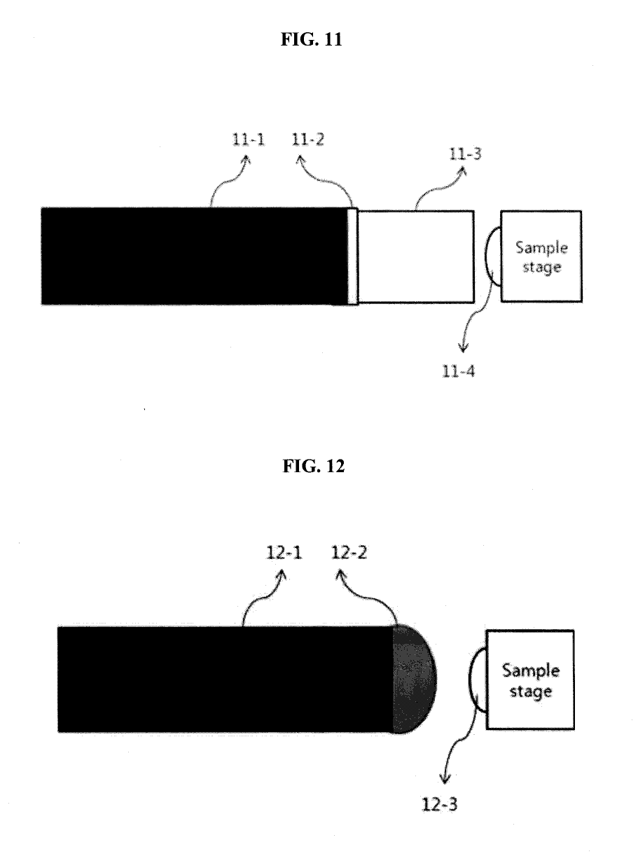

Exemplary embodiments of FIGS. 11, 12, 13, 14, 15, and 16 are schematic diagrams of probes shown in an optical coherent fiber bundle of a sample stage in an optical coherence imaging system using the optical coherent fiber bundle as the endoscopic probe. FIG. 11 is a schematic diagram of a probe which can be adopted at the end of the optical coherent fiber bundle of the sample stage in the optical coherent fiber bundle of a sample stage in an optical coherence imaging system using the optical coherent fiber bundle as the endoscopic probe and shows an exemplary embodiment in which a green lens 11-3 is attached to the end of the optical coherent fiber bundle 101. By substituting the optical coherent fiber bundle constituting the sample stage of FIGS. 1 and 2 with FIG. 11, the light reflected or scattered on the sample is more efficiently focused to increase the intensity of the interference spectrum signal.

FIG. 12 is a schematic diagram of a probe which can be adopted in the end of the optical coherent fiber bundle of the sample stage in the optical coherence imaging system using the optical coherent fiber bundle as the endoscopic probe and shows an exemplary embodiment in which an optical fiber integrated lens 12-2 is formed at a front end of the optical coherent fiber bundle in order to focus or collect a larger light amount on the end of the optical fiber end of the sample stage. By substituting the optical coherent fiber bundle constituting the sample stage of FIGS. 1 and 2 with FIG. 12, the light reflected or scattered on the sample is more efficiently focused to increase the intensity of the interference spectrum signal.

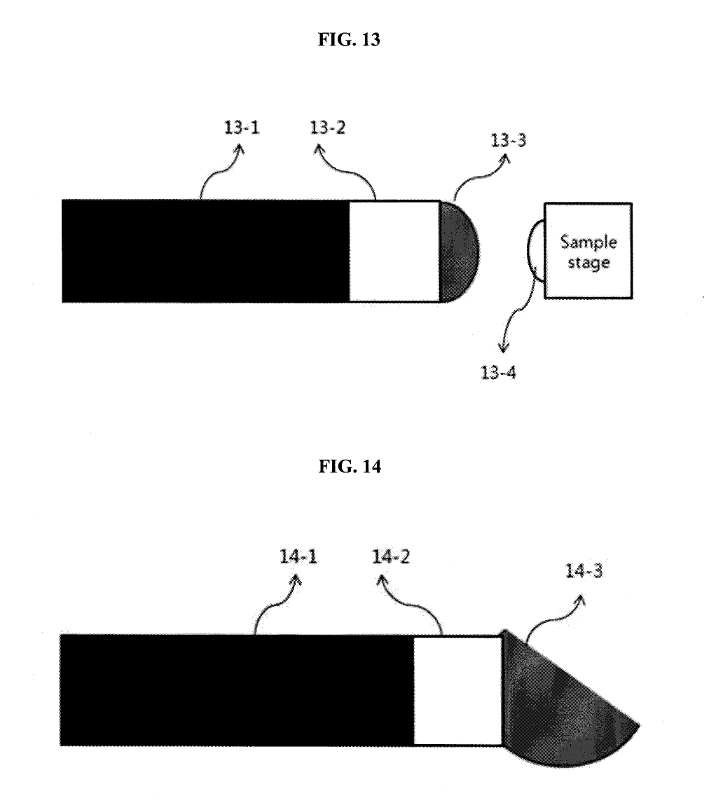

FIG. 13 is a schematic diagram of a probe which can be adopted in the end of the optical coherent fiber bundle of the sample stage in the optical coherence imaging system using the optical coherent fiber bundle as the endoscopic probe and shows an exemplary embodiment in which a coreless silica fiber (CSF) 13-2 is coupled to the front end of the optical coherent fiber bundle by using an optical fusion connection method and an optical fiber integrated lens 13-3 is formed at a front end of the CSF in order to focus or collect a larger light amount on the end of the optical fiber end of the sample stage. By substituting the optical coherent fiber bundle constituting the sample stage of FIGS. 1.2 and 2 with FIG. 13, the light reflected or scattered on the sample is more efficiently focused to increase the intensity of the interference spectrum signal.

FIG. 14 is a schematic diagram of a probe which can be adopted in the end of the optical coherent fiber bundle of the sample stage in the optical coherence imaging system using the optical coherent fiber bundle as the endoscopic probe and shows an exemplary embodiment in which an optical fiber integrated lens 14-3 cut vertically to enable side image is formed at a front end of the optical coherent fiber bundle in order to focus or collect a larger light amount on the end of the optical fiber end of the sample stage. By substituting the optical coherent fiber bundle constituting the sample stage of FIGS. 1.2 and 2 with FIG. 14, the light reflected or scattered on the sample is more efficiently focused to increase the intensity of the interference spectrum signal and enable the side imaging.

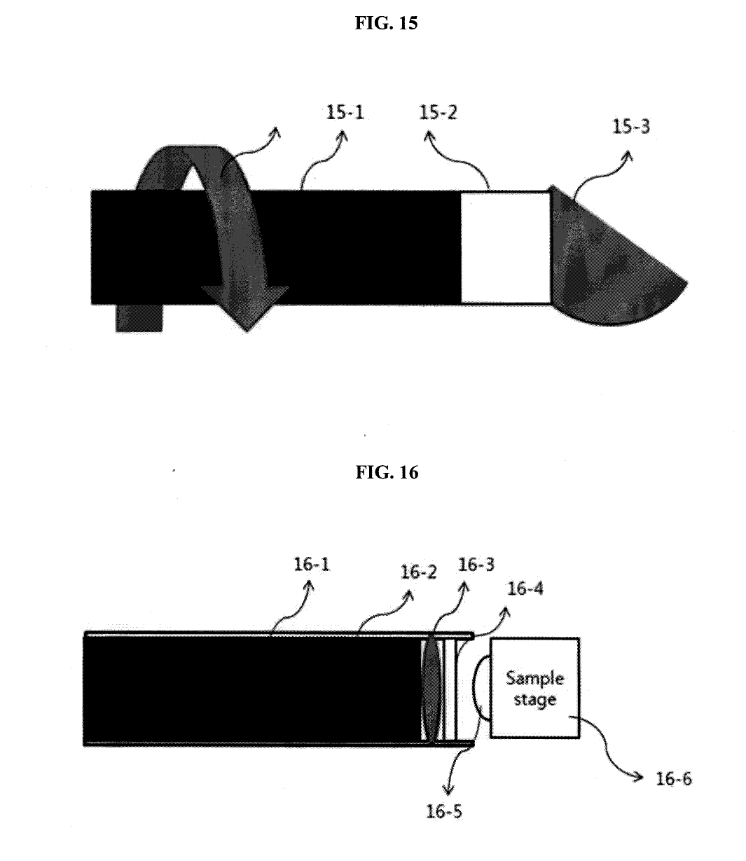

FIG. 15 is a schematic diagram of a probe which can be adopted in the end of the optical coherent fiber bundle of the sample stage in the optical coherence imaging system using the optical coherent fiber bundle as the endoscopic probe and shows an exemplary embodiment in which the probe of FIG. 14 in which a 3D image is implemented by enabling the rotation of an optical fiber integrated lens 15-3 cut vertically to enable side image is formed at the front end of the optical coherent fiber bundle in order to focus or collect a larger light amount on the end of the optical fiber end of the sample stage. By substituting the optical coherent fiber bundle constituting the sample stage of FIGS. 1 and 2 with FIG. 15, the light reflected or scattered on the sample is more efficiently focused to increase the intensity of the interference spectrum signal and enable the side imaging. In addition, it has an advantage that a rotary 3D image can be formed.

FIG. 16 shows another exemplary embodiment according to the present invention like FIG. 11 with showing a cross-sectional view in which a micro-lens is attached to the end of the optical coherent fiber bundle and thereafter, packaged to be suitable for the system. Therefore, by substituting the optical coherent fiber bundle of the sample stage of the present invention (FIG. 1) with FIG. 16, the light-reflected or scattered on the sample is more efficiently focused to increase the intensity of the interference spectrum signal. The exemplary embodiments of FIGS. 11, 12, 13, 14, 15, and 16 can simplify a system configuration and ease the optical alignment, and further, the exemplary embodiments can be usefully used even in the human body.

What is claimed is:

1. An optical coherence tomography image acquiring method for acquiring a tomography image of a sample surface and an internal structure based on an optical fiber bundle, comprising:
splitting and irradiating a light source having a predetermined bandwidth based on a center wavelength into a fixed reference stage and a sample stage constituted by an optical fiber bundle through an optical splitter;
generating an interference signal after light reflected on a mirror of the reference stage and light reflected on the sample through the optical splitter again through the optical filter bundle meet each other again;
perform 1D lateral scan with respect to an incident surface of the sample stage constituted by the optical fiber bundle in order to acquire 2D image information on the sample and detecting interference signals generated from light reflected on the sample surface and an internal tomography interface layer by using a spectrometer of a detection stage and a line CCD camera; and
acquiring a tomography image on after signal processing the detected interference signals and outputting the acquired tomography image onto a monitor as a video.

2. An optical coherence tomography image acquiring method for acquiring a tomography image of a sample surface and an internal structure based on an optical fiber bundle, comprising:
irradiating light of a light source having a predetermined bandwidth based on a center wavelength into an integrated stage of a reference stage and a sample stage constituted by an optical fiber bundle through an optical splitter of which one-side port is blocked;
generating an interference signal after light reflected on an emissions surface of the optical fiber bundle and light reflected on the sample through the optical splitter again through the optical filter bundle;
perform 1D lateral scan with respect to an incident surface of the sample stage constituted by the optical fiber bundle in order to acquire 2D image information on the sample and detecting interference signals generated from light reflected on the sample surface and an internal tomography interface layer by using a spectrometer of a detection stage and a line CCD camera; and
acquiring a tomography image on after signal processing the detected interference signals and outputting the acquired tomography image onto a monitor as a video.

3. The optical coherence tomography image acquiring method of claim 2, wherein light is irradiated by using an optical circulator instead of the optical splitter.

4. The optical coherence tomography image acquiring method of claim 1, wherein the sample stage constituted by the optical fiber bundle serves a small-sized endoscopic probe.

5. The optical coherence tomography image acquiring method of claim 2, wherein the sample stage constituted by the optical fiber bundle serves a small-sized endoscopic probe.

6. The optical coherence tomography image acquiring method of claim 1, wherein the optical fiber bundle has a diameter in the range of 0.4 to 2 mm and 10000 to 100000 cores are focused on one cladding.

7. The optical coherence tomography image acquiring method of claim 2, wherein the optical fiber bundle has a diameter in the range of 0.4 to 2 mm and 10000 to 100000 cores are focused on one cladding.

8. The optical coherence tomography image acquiring method of claim 4, wherein the cores are arranged at regular intervals with the distance between the cores of 4 μm or less to be focused.

9. The optical coherence tomography image acquiring method of claim 5, wherein the cores are arranged at regular intervals with the distance between the cores of 4 μm or less to be focused.

10. The optical coherence tomography image acquiring method of claim 1, wherein the sample stage constituted by the optical fiber bundle is surrounded by a jacket for protecting the optical fiber bundle.

11. The optical coherence tomography image acquiring method of claim 2, wherein the sample stage constituted by the optical fiber bundle is surrounded by a jacket for protecting the optical fiber bundle.

12. The optical coherence tomography image acquiring method of claim 1, wherein the optical fiber bundle transfers an image projected onto an optical fiber bundle incident surface to an optical fiber bundle emission surface without the distortion of the image.

13. The optical coherence tomography image acquiring method of claim 2, wherein the optical fiber bundle transfers an image projected onto an optical fiber bundle incident surface to an optical fiber bundle emission surface without the distortion of the image.

14. The optical coherence tomography image acquiring method of claim 1, wherein parallel light generated by a beam balancer in the sample stage is focused on one core of the optical fiber bundle by using an objective lens.

15. The optical coherence tomography image acquiring method of claim 2, wherein parallel light generated by a beam balancer in the sample stage is focused on one core of the optical fiber bundle by using an objective lens.

16. The optical coherence tomography image acquiring method of claim 1, wherein the parallel light generated by the beam balancer in the sample stage is scanned on a lateral axis by using a uniaxial Galvano scanner mirror.

17. The optical coherence tomography image acquiring method of claim 2, wherein the parallel light generated by the beam balancer in the sample stage is scanned on a lateral axis by using a uniaxial Galvano scanner mirror.

18. The optical coherence tomography image acquiring method of claim 1, wherein the parallel light generated by the beam balancer in the sample stage is scanned on a longitudinal axis and the lateral axis by using a biaxial Galvano scanner mirror.

19. The optical coherence tomography image acquiring method of claim 2, wherein the parallel light generated by the beam balancer in the sample stage is scanned on a longitudinal axis and the lateral axis by using a biaxial Galvano scanner mirror.

20. The optical coherence tomography image acquiring method of claim 1, wherein the parallel light generated by the beam balancer in the sample stage is scanned on the lateral axis by using a uniaxial linear feeding apparatus.

21. The optical coherence tomography image acquiring method of claim 2, wherein the parallel light generated by the beam balancer in the sample stage is scanned on the lateral axis by using a uniaxial linear feeding apparatus.

22. The optical coherence tomography image acquiring method of claim 1, wherein the parallel light generated by the beam balancer in the sample stage is scanned on the lateral axis by using a biaxial linear feeding apparatus.

23. The optical coherence tomography image acquiring method of claim 2, wherein the parallel light generated by the beam balancer in the sample stage is scanned on the lateral axis by using a biaxial linear feeding apparatus.

24. The optical coherence tomography image acquiring method of claim 1, wherein scanning is performed in the sample stage by using an optical switch and a coupler.

25. The optical coherence tomography image acquiring method of claim 2, wherein scanning is performed in the sample stage by using an optical switch and a coupler.

26. The optical coherence tomography image acquiring method of claim 1, wherein scanning is performed in the sample by using the optical switch and an optical circulator.

27. The optical coherence tomography image acquiring method of claim 2, wherein scanning is performed in the sample by using the optical switch and an optical circulator.

28. The optical coherence tomography image acquiring method of claim 1, wherein a green lens is attached to the end of the optical fiber bundle.

29. The optical coherence tomography image acquiring method of claim 2, wherein a green lens is attached to the end of the optical fiber bundle.

30. The optical coherence tomography image acquiring method of claim 1, wherein an optical fiber integrated is formed at a front end of the optical fiber bundle.

31. The optical coherence tomography image acquiring method of claim 2, wherein an optical fiber integrated is formed at a front end of the optical fiber bundle.

32. The optical coherence tomography image acquiring method of claim 1, wherein a coreless silica fiber is coupled to a front end of the optical fiber bundle by using an optical fusion connection method and the optical fiber integrated lens is formed at a front end of the CSF.

33. The optical coherence tomography image acquiring method of claim 2, wherein a coreless silica fiber is coupled to a front end of the optical fiber bundle by using an optical fusion connection method and the optical fiber integrated lens is formed at a front end of the CSF.

34. The optical coherence tomography image acquiring method of claim 1, wherein the optical fiber integrated lens is vertically cut to enable side imaging.

35. The optical coherence tomography image acquiring method of claim 2, wherein the optical fiber integrated lens is vertically cut to enable side imaging.

36. The optical coherence tomography image acquiring method of claim 1, wherein a focusing lens is attached to the end of the optical fiber bundle.

37. The optical coherence tomography image acquiring method of claim 2, wherein a focusing lens is attached to the end of the optical fiber bundle.
